(19) United States
(12) Patent Application Publication
(10) Pub. No.: US 2012/0059069 A1
(43) Pub. Date: Mar. 8, 2012

(54) AMPHIPHILIC BRANCHED COPOLYMER, METHODS OF PREPARATION, EMULSIONS, AND USES

(75) Inventors: Paul Hugh Findlay, Liverpool (GB); Brodyck James Lachlan, Liverpool (GB); Neil John Simpson, Liverpool (GB); Sharon Todd, Liverpool (GB); Roselyne Marie Andree Baudry, Liverpool (GB); Steven Paul Rannard, Liverpool (GB); Jonathan Victor Mark Weaver, Liverpool (GB)

(73) Assignee: UNILEVER PLC, London (GB)

(21) Appl. No.: 13/266,745

(22) PCT Filed: Apr. 28, 2010
(86) PCT No.: PCT/GB2010/000845

§ 371 Date: Oct. 27, 2011

(30) Foreign Application Priority Data
Apr. 28, 2009 (GB) 0907273.7

(51) Int. Cl. A61K 47/32; A23D 7/005; C08L 39/00; C08F 236/20; A61K 8/81

(52) U.S. Cl. 514/772.6; 526/312; 524/555; 426/602

(57) ABSTRACT

The present invention relates to an amphiphilic branched copolymer obtainable by an addition polymerisation process, the method of production of same and uses thereof as emulsions wherein said polymer comprises:
at least two chains which are covalently linked by a bridge other than at their ends; and wherein the at least two chains comprise at least one ethylenically monounsaturated monomer, and wherein the bridge comprises at least one ethylenically polyunsaturated monomer; and wherein
the polymer comprises a residue of a chain transfer agent and optionally a residue of an initiator, and wherein
at least one of the monounsaturated monomer(s) and polyunsaturated monomer(s) and chain transfer agent(s) is a hydrophilic residue; and
at least one of one of the monounsaturated monomer(s) and polyunsaturated monomer(s) and chain transfer agent(s) is a hydrophobic residue, and wherein
the mole ratio of polyunsaturated monomer(s) to monounsaturated monomer(s) is in a range of from 1:100 to 1:4, and wherein
at least one of the monounsaturated monomer(s) and polyunsaturated monomer(s) and chain transfer agent(s) comprises a moiety that is capable of responding to an external stimuli thereby creating a physical or chemical change in the solubility of the said moiety; and wherein
the air-water surface tension of the polymer changes from between 35 mN/m to 60 mN/m upon application of the external stimulus.

### CROSS-REFERENCE TO RELATED APPLICATIONS

This application is the national phase entry of PCT Application No. PCT/GB2010/000845, filed Apr. 28, 2010, which claims priority to GB Application No. 0907273.7, filed Apr. 28, 2009. The disclosures of said applications are hereby incorporated herein by reference.

### BACKGROUND

The present invention relates to branched copolymers, more specifically certain amphiphilic branched copolymers, methods for their preparation, compositions comprising such copolymers and their use as responsive emulsifiers. The copolymers are especially responsive in nature and form extremely stable emulsions which can be tuned to demulsify upon application of external stimuli.

Branched polymers are polymer molecules of a finite size which are branched. Branched polymers differ from crosslinked polymer networks which tend towards an infinite size having interconnected molecules and which are generally not soluble. In some instances, branched polymers have advantageous properties when compared to analogous linear polymers. For instance, solutions of branched polymers are normally less viscous than solutions of analogous linear polymers. Moreover, higher molecular weights of branched copolymers can be solubilised more easily than those of corresponding linear polymers. In addition, as branched polymers tend to have more end groups than a linear polymer they generally exhibit strong surface-modification properties. Thus, branched polymers are useful components of many compositions and are therefore utilised in a variety of applications.

Branched polymers are usually prepared via a step-growth mechanism via the polycondensation of suitable monomers and are usually limited via the chemical functionality of the resulting polymer and the molecular weight. In addition polymerisation, a one-step process can be employed in which a multifunctional monomer is used to provide functionality in the polymer chain from which polymer branches may grow. However, a limitation on the use of conventional one-step processes is that the amount of multifunctional monomer must be carefully controlled, usually to substantially less than 0.5% w/w in order to avoid extensive cross-linking of the polymer and the formation of insoluble gels. It is difficult to avoid cross-linking using this method, especially in the absence of a solvent as diluent and/or at high conversion of monomer to polymer.

Amphiphilic branched copolymers are branched copolymers which have nominally a hydrophilic portion and a hydrophobic portion, this can be either a permanent or transient hydrophilic or hydrophobic moiety; for example a weak acid or basic unit wherein the hydrophobicity is dependant on the pH of the polymer solution.

It has now been found that amphiphilic branched non-cross-linked copolymers having novel polymer architecture can be prepared by an addition polymerisation method. These amphiphilic branched copolymers can form stable emulsions and their composition and architecture can be tuned such that the formed emulsions can be triggered to demulsify upon a change in the external environment. These copolymers and emulsions can be readily synthesised and have a variety of applications as a result of their advantageous properties.

For example, many cosmetic, pharmaceutical, agrochemical, electronic, medical, diagnostic, coatings or food products are in the form of emulsions, either as a dispersed hydrophobic phase in a continuous phase (oil-in-water (o/w)), or as a hydrophilic phase dispersed in a continuous hydrophobic phase (water-in-oil (w/o)). The formation of stable emulsions requires the use of materials that can adsorb at the biphasic interface and prevent coalescence, or demulsification, of the droplets. Amphiphilic molecules such as surfactants or surface-active polymers are typically used for the stabilisation of oil and water emulsions as one part of the surfactant interacts with the oil phase and the other interacts with the water phase. These types of emulsions have considerable disadvantages such as their kinetic instability, high foaming, requiring large amounts of emulsifiers or cosurfactants and irritancy due to the surfactant molecules, to name but a few.

Emulsions stabilised with inorganic or organic particles have been shown to have excellent stability with low foaming and reduced irritancy. Typically, these emulsions are formed by the use of finely divided inorganic particles such as silica, alumina or metal oxides and the like. The driving force for particles stabilising an interface is the reduction in free energy as the particle adsorbs. In many cases particle-stabilised emulsions are extremely stable as the energy required to remove the particle from the surface is large, in some instances, the particles that stabilise an emulsion droplet can be considered to be irreversibly adsorbed. Such particles are referred to as particulate, Pickering or Ramsden emulsifiers and are commonly inorganic species. Organic particles have also been investigated as Pickering emulsifiers.

Hydrophobic actives, such as drugs, agrochemicals and fragrances, dyes, or reactive actives are often only utilisable if they can be stabilised in hydrophilic environments for sustained periods of time, such as in the body, as a concentrate or in aqueous domestic and personal care formulations. Consequently significant efforts have been made towards developing suitable vehicles for such actives. In this context, self-assembled polymer structures, such as micelles, have received significant attention due to their functionality and size. Encapsulation of actives within these polymeric vehicles followed by their controlled and/or triggered release has been routinely used as a test for their suitability.

WO 99/46301 discloses a method of preparing a branched polymer comprising the steps of mixing together a monofunctional vinylic monomer with from 0.3 to 100% w/w (of the weight of the monofunctional monomer) of a multifunctional vinylic monomer and from 0.0001 to 50% w/w (of the weight of the monofunctional monomer) of a chain transfer agent and optionally a free-radical polymerisation initiator and thereafter reacting said mixture to form a copolymer. The examples of WO 99/46301 describe the preparation of primarily hydrophobic polymers and, in particular, polymers wherein methyl methacrylate constitutes the monofunctional monomer. These polymers are useful as components in reducing the melt viscosity of linear poly(methyl methacrylate) in the production of moulding resins.

WO 99/46310 discloses a method of preparing a (meth)acrylate functionalised polymer comprising the steps of mixing together a monofunctional vinylic monomer with from 0.3 to 100% w/w (based on monofunctional monomer) of a polyfunctional vinylic monomer and from 0.0001 to 50% w/w of a chain transfer agent, reacting said mixture to form a polymer and terminating the polymerisation reaction before 99% conversion. The resulting polymers are useful as components of surface coatings and inks, as moulding resins or in curable compounds, for example curable moulding resins or photoresists.

WO 02/34793 discloses a rheology modifying copolymer composition containing a branched copolymer of an unsaturated carboxylic acid, a hydrophobic monomer, a hydrophobic chain transfer agent, a cross-linking agent, and, optionally, a steric stabilizer. The copolymer provides increased viscosity in aqueous electrolyte-containing environments at elevated pH. The method for production is a solution polymerisation process. The polymer is lightly cross-linked, less than 0.25%.

H. Hayashi et al. (Macromolecules 2004, 37, 5389-5396) describe the emulsion polymerisation of 2-(diethylamino)ethyl methacrylate to obtain gel particulates in the size range between 50 and 680 nm in the presence of a cross-linking agent such as ethylene glycol dimethacrylate, using alpha-vinylbenzyl-omega-carboxy-PEG as a stabilising reagent. A chain transfer agent is not utilised in the polymerisation process. These nanogels can potentially be utilitised in applications such as diagnostics and controlled drug releasing devices.

U.S. Pat. No. 6,361,768 B1 discloses a hydrophilic ampholytic polymer synthesised by reacting polymerisable amino and carboxy-functional ethylenically unsaturated monomers together with a non-ionic hydrophilic monomer, to provide a polymer having a glass transition temperature above about 50° C., and optionally hydrophobic monomer(s), and cross-linking monomer(s), however without the use of a chain transfer agent. The copolymer is precipitated from a polymerisation media which includes a suitable organic solvent. The polymer is optionally lightly cross-linked. The resulting copolymer is in the form of a fine powder, with submicron particle size. As such it is suitable for use as a thickener or rheology modifier in personal care formulations, as a bioadhesive, and for pharmaceutical applications. The ampholytic nature is probably a consequence of the designed compatibility with high salt/surfactant levels.

US 2006/0106133 A1 discloses an ink-jet ink comprising an amphiphilic polymer, wherein the polymer comprises hydrophilic and hydrophobic portions, at a molecular weight range from 300 to 100,000 Daltons, and may be in the form of a straight chain polymer, a star-form polymer or an emulsion form having a polymer core. A chain transfer agent is not used in the production of the polymer. The polymer is used as a wetting aid in the formation of uniform ink droplets on the substrate.

EP 1 384 771 A1 discloses acid-functional triggered responsive polyelectrolytes, that are stable and insoluble in an aqueous system at relatively high ionic strength or base concentration and that disperse, disintegrate, dissolve, destabilise, swell, or combinations thereof, when the ionic strength or base strength of the aqueous system changes (notably decreases). The polyelectrolytes thus show a triggered response. The polyelectrolyte is one or more alkali soluble polymers comprising: (a) 5 to 70 weight percent of acidic monomers selected from for example (meth)acrylic acid, (b) 30 to 95 weight percent of one or more non-ionic vinyl monomers selected from for example butyl acrylate and methyl methacrylate, and optionally (c) 0.01 to 5 weight percent of one or more cross-linking agents such as polyethylenically unsaturated monomers or a metal cross-linking agent. The polymers are prepared via an emulsion polymerisation route cross-linked with either a polyvalent metal salt (like zinc and calcium) or a polyvinylic monomer, prepared either with or without a chain transfer agent, to reduce the molecular weight of the polymer. The triggered response may lead to release of components that are entrapped within the polyelectrolytes. The disclosure does not embody hydrogen-bonding and is based on alkali-swellable cross-linked polymers, high pH being the swelling trigger.

WO 2008/004988 discloses an amphiphilic linear copolymer, having at least one hydrophobic endgroup. The first monomer is such that the copolymer is thermally responsive and the second monomer comprises a carboxylic acid or carboxylate group. The copolymer is arranged in micelles in a liquid, and the liquid may be an organic liquid, whereby the micelles adopt a core-shell structure in which a hydrophilic core is surrounded by a hydrophobic shell. The micelles may contain a biologically active compound (for example an enzyme) which may be released from the micelle by temperature increase. The copolymer is not branched or cross-linked. The micelles may be thermally responsive micelles, the thermoresponsive nature of these polymers is derived from the lower critical solution temperature (LCST) of the N-alkyl acrylamide monomers used in their preparation, in particular N-isopropyl acrylamide. The polymers contain a carboxylic acid-containing second monomer.

U.S. Pat. No. 7,316,816 B2 discloses temperature and pH sensitive amphiphilic linear copolymers. The copolymers comprise at least three types of monomeric units: a temperature-sensitive monomer, a hydrophilic monomer, and a hydrophobic monomer comprising at least one pH-sensitive moiety; wherein said hydrophobic monomeric unit is derived from a copolymerisable unsaturated fatty acid. The molecular weight may be reduced by the use of a chain transfer agent. The copolymers can be arranged into core-shell structures with a hydrophobic core, wherein the core may contain a hydrophobic (pharmaceutically) active ingredient. Upon change of the external conditions (for example temperature or pH), the entrapped ingredient can be released.

WO 2008/019984 discloses amphiphilic linear block copolymers, a process for making the same, and its use in emulsions. The block copolymers comprise a hydrophilic block and a hydrophobic block and can be used as an emulsifier or as a co-emulsifier, particularly in water-in-oil emulsions. The polymers are composed of N-vinyl pyrrolidone/N-alkyl acrylamine copolymerised with an alkyl(meth)acrylate.

US 2004/0052746 A1 discloses polymers that are amino-functional terpolymers to produce the necessary association at the desired pH range. The polymers are the product of a monomer mixture comprising at least one amino-substituted vinyl monomer; at least one non-ionic vinyl monomer; at least one associative vinyl monomer; at least one semi-hydrophobic vinyl surfactant monomer; and, optionally, comprising one or more hydroxy-substituted non-ionic vinyl monomers, polyunsaturated cross-linking monomer (when present, then at a most preferred concentration of 0.1 to 1 wt % of the monomer mixture), chain transfer agent (when present then at a concentration of at least 0.1 wt % of the monomer mixture), or polymeric stabilizer. These vinyl addition polymers have a combination of substituents, including amino substituents that provide cationic properties at low pH, hydrophobic substituents, hydrophobically modified polyoxyalkylene substituents, and hydrophilic polyoxyalkylene substituents. The polymers are rheology modifiers, by increase of viscosity when applied in emulsions at low pH, and are compatible with cationic materials.

US 2006/0183822 A1 discloses an ampholytic copolymer, and polyelectrolyte complexes which comprise such an ampholytic copolymer, and to cosmetic or pharmaceutical compositions which comprise at least one ampholytic copolymer or one polyelectrolyte complex. The copolymer is composed of a balanced proportion of anionic/cationic monomers, an amide-containing polymer, a hydrophobic monomer, and optionally a cross-linker (for example a diethylenically unsaturated compound), and/or a chain transfer agent. The polymers are rheology modifiers (thickeners) and film-form in personal care applications.

WO 2002/047665 discloses a method for stabilising emulsions (water-in-oil or oil-in-water) by polymer particles that will adhere to the interface of the droplets. The solid particles have a size of approximately 1 micrometer. The emulsion droplets can be further stabilised by some form of cross-linking between the particles, for example by a sintering process. Emulsions are formed via the use of cross-linked polymer beads; the beads can then be further reacted to give a hard shell by ionic interactions with a suitable polyelectrolyte. The polymers are not soluble and branched and do not show responsive behaviour upon changing conditions.

GB 2 403 920 A discloses the use of particulates (diameter preferably 0.05 to 5 μm) as Pickering emulsifiers in an oil-in water or water-in-oil emulsion. The particulates comprise at least one polymer (latex), wherein the hydrophilic/hydrophobic balance of the polymer can be varied on application of a stimulus (for example pH change from a pH above the pKa of the polymer to a pH below the pKa of the polymer) to break the emulsion or to cause phase inversion. A chain transfer agent is not used in the production of the polymers.

EP 1 726 600 A1 discloses compositions comprising an oil phase, an aqueous phase, at least one emulsifying system of water-in-oil type, optionally at least one emulsifying system of oil-in-water type, in the form of an inverse latex comprising from 20% to 70% by mass of a branched or cross-linked polyelectrolyte. The polyelectrolyte is a copolymer of 2-acrylamido-2-methylpropanesulfonic acid partially or totally salified with N,N-dimethlacrylamide and optionally one or more monomers chosen from monomers containing a partially or totally salified weak acid function and/or from neutral monomers other than N,N-dimethylacrylamide. The polyelectrolytes may be cross-linked by a multifunctional monomer, and a chain transfer agent is not used in the production process of the polymers. These polymers are used as emulsifiers and thickeners in cosmetic or pharmaceutical compositions, and increase in viscosity when salt is added to the solution.

Koh and Saunders (Chem. Commun (2000) 2461) discloses oil-in-water (o/w) emulsions (1-bromohexadecane in water) exhibiting reversible thermally induced gelation, wherein the emulsifier is a linear graft (comb) copolymer containing poly(N-isopropylacrylamide) as the backbone and pendant poly(ethylene glycol) methacrylate groups (average number molecular weight of 360). The polymer is produced using a free radical polymerisation process. Increasing the temperature to a value above the lower critical solution temperature of the polymer led to a strong increase of the viscosity of the emulsion due to gelation. The reversibility of the process was demonstrated by decreasing the temperature to below 50° C., leading to a strong decrease of the viscosity. The emulsion did not break up on temperature decrease, and some residual flocs of agglomerated emulsion droplets were still present.

U.S. Pat. No. 6,528,575 B1 discloses cross-linked acid-functionalised copolymers obtainable by precipitation polymerization of monomer mixtures, comprising (a) monoethylenically unsaturated C3-C8 carboxylic acids, their anhydrides or mixtures of said carboxylic acids and anhydrides, (b) compounds with at least 2 non-conjugated ethylenic double bonds in the molecule as cross-linkers and possibly (c) other monoethylenically unsaturated monomers which are copolymerizable with monomers (a) and (b), in the presence of free-radical polymerization initiators and from 0.1 to 20% by weight, based on the monomers used, of saturated, non-ionic surface-active compounds. These polymers are cross-linked, and produced via a precipitation route without the presence of a chain transfer agent. The polymers are used as stabiliser in oil-in-water emulsions in amounts of from 0.01 to 5% of the weight of the emulsions. Cosmetic and pharmaceutical formulations based on oil-in-water emulsions which contain said precipitation polymers are also disclosed. The polymers are non-hydrogen bonding (non-associative).

U.S. Pat. No. 6,020,291 discloses aqueous metal working fluids used as lubricant in metal cutting operations. The fluids contain a mist suppressing branched copolymer, including hydrophobic and hydrophilic monomers, and optionally a monomer comprising two or more ethylenically unsaturated bonds. Optionally, the metal working fluid may be an oil-in-water emulsion. The polymers are based on poly(acrylamides) containing sulfonate-containing and hydrophobically modified monomers. The polymers are cross-linked to a very small extent by using very low amount of bis-acrylamide, without using a chain transfer agent.

Armes et al (J. Mat. Chem. (2008) 18, 545-552) discloses a pH-responsive, non-soluble, cross-linked polymer latex prepared using a 2-vinyl pyridine monomer utilizing divinyl benzene prepared via an emulsion polymerisation route. The latex particles can act as efficient emulsion stabilisers capable of responding to a reduction in solution pH resulting in demulsification.

WO 2008/071660 discloses branched polymers which are slightly basic. At low pH these polymers are protonated and soluble in water. Upon increase of pH the basic residues of the polymer are deprotonated and therewith become more hydrophobic. Due to the hydrophobic groups the polymer collapses into a hydrophobic core surrounded by a hydrophilic shell, comprising ethylene oxide groups, forming a small particle. The hydrophilic shell maintains the particles in solution, and these particles can be used as Pickering emulsifiers.

A disadvantage of the use of linear polymers according to prior art, is that they do not sufficiently stabilise emulsions, or linear co-polymers are required to be synthesised in a controlled manner in order to afford block-like structures. This makes the production process complex. Moreover many polymers that are cross-linked rather than branched are microgels cross-linked to have a large molecular weight, and consequently they do not truly dissolve, and are difficult to process. Consequently, this may lead to the polymers acting as rheology modifiers by increasing solution viscosity, which can be disadvantageous.

### SUMMARY

Therefore it is an object of the present invention to provide polymeric emulsifiers that can be used to stabilise emulsions, without increasing the viscosity of the solution. A further object is to provide emulsions which contain functional ingredients in the dispersed phase, and wherein the emulsion is stable upon storage. Upon a change of the external conditions by way of one or more stimuli the functional ingredients may be released from the dispersed phase. A further object of the present invention is to provide concentrated stable emulsions, wherein the concentration of the dispersed phase is high.

Additionally, once formed a subsequent reaction may be employed whereby a further cross-linking reaction occurs resulting in emulsion droplet encapsulation via one or more inter or intramolecular reactions within the polymeric emulsifier.

It has now been found by the inventors that certain amphiphilic branched polymers are able to efficiently stabilise emulsions and their composition and architecture can be tuned such that they can be triggered to demulsify in a controlled manner. The amphiphilic branched polymers comprises residues of a monounsaturated monomer, a polyunsaturated monomer, and a chain transfer agent. The polymers according to the invention may be used as emulsifiers. Upon a change in external conditions, for example, the solution pH, salt concentration or the temperature, the emulsion droplets may demulsify releasing the emulsified phase into the bulk phase.

The branched polymers are therefore capable of demulsification by, for example, a change in the solution pH. The emulsion can thus be considered to be a responsive emulsion, due to the response of the polymer under the changing conditions.

In one embodiment the emulsions comprising the polymers may therefore demulsify in response to external changes, therewith releasing a compound trapped in the dispersed phase. The chemical composition, architecture and molecular weight of the copolymer can be controlled during the polymerisation step thereby allowing the degree of demulsification in the formed emulsion to be controlled. In this way a method is provided by which a controlled emulsification and disassembly of emulsion droplets can be achieved. The responsive behaviour is due to interaction of the copolymer with the oil-water interface, depending on the external conditions like pH or temperature.

Therefore according to a first aspect of the present invention there is provided an amphiphilic branched copolymer obtainable by an addition polymerisation process, wherein said polymer comprises:
at least two chains which are covalently linked by a bridge other than at their ends; and wherein the at least two chains comprise at least one ethylenically monounsaturated monomer, and wherein the bridge comprises at least one ethylenically polyunsaturated monomer; and wherein
the polymer comprises a residue of a chain transfer agent and optionally a residue of an initiator, and wherein
at least one of the monounsaturated monomer(s) and polyunsaturated monomer(s) and chain transfer agent(s) is a hydrophilic residue; and
at least one of one of the monounsaturated monomer(s) and polyunsaturated monomer(s) and chain transfer agent(s) is a hydrophobic residue, and wherein
the mole ratio of polyunsaturated monomer(s) to monounsaturated monomer(s) is in a range of from 1:100 to 1:4, and wherein
at least one of the monounsaturated monomer(s) and polyunsaturated monomer(s) and chain transfer agent(s) comprises a moiety that is capable of responding to an external stimuli thereby creating a physical or chemical change in the solubility of the said moiety; and wherein
the air-water surface tension of the polymer changes from between 35 mN/m to 60 mN/m upon application of the external stimulus.

It is preferred that for the amphiphilic branched copolymers according to the first aspect of the present invention the air-water surface tension of the polymer changes from between 40 mN/m to 55 mN/m upon application of an external stimulus. More preferably the air-water surface tension of the polymer changes from between 42 mN/m to 52 mN/m upon application of the external stimulus.

The external stimulus to which the amphiphilic branched copolymers according to the first aspect of the present invention may react include but are not limited to pH, ionic strength, sonic means, temperature, concentration, electromagnetic radiation, or the addition of a further chemical entity.

It is also preferred that for the amphiphilic branched copolymers according to the present invention one or more of the monounsaturated monomer(s), polyunsaturated monomer(s) and chain transfer agent(s) are each individually responsive to the external stimuli.

In some cases, the amphiphilic branched copolymer comprises a monofunctional monomer which comprises one or more monomers selected from the group consisting of: vinyl acids, vinyl acid esters, vinyl aryl compounds, vinyl acid anhydrides, vinyl amides, vinyl ethers, vinyl amines, vinyl aryl amines, vinyl nitriles, vinyl ketones, and derivatives thereof and corresponding allyl variants thereof; hydroxyl-containing monomers and monomers which may be post-reacted to form hydroxyl groups, acid-containing or acid-functional monomers; zwitterionic monomers and quaternised amino monomers; oligomeric, polymeric and di- and multi-functionalised monomers.

In some cases, the monofunctional monomer are vinylic or allylic and are selected from the group consisting of styrenics, acrylics, methacrylics, allylics, acrylamides, methacrylamides, vinyl acetates, allyl acetates, N-vinyl amines, allyl amines, vinyl ethers, and allyl ethers.

In some embodiments, the amphiphilic branched copolymer comprises a monofunctional monomer, wherein when the monofunctional monomer provides the necessary hydrophilicity in the copolymer, the monofunctional monomer is a residue of a hydrophilic monofunctional monomer, comprising a molecular weight of at least 1000 Daltons.

In some embodiments, the amphiphilic branched copolymer comprises a multifunctional monomer which is selected from the group consisting of: divinyl aryl monomers; (meth)acrylate diesters; polyalkylene oxide di(meth)acrylates; divinyl (meth)acrylamides; divinyl ethers; and tetra- or tri-(meth)acrylate esters; vinyl or allyl esters, amides or ethers of pre-formed oligomers or polymers formed via ring-opening polymerisation, and oligomers or polymers formed via a living polymerisation technique such as oligo- or poly(1,4-butadiene).

According to a second aspect of the present invention there is provided a method of preparing an amphiphilic branched copolymer according to the first aspect of the present invention by way of an addition polymerisation process, which comprises the mixing together of:
(a) at least one ethylenically monounsaturated monomer;
(b) from 1 to 25 mole % (based on the number of moles of monofunctional monomer(s)) of at least one ethylenically polyunsaturated monomer;
(c) a chain transfer agent; and
(d) an initiator, and subsequently reacting said mixture to form a branched copolymer.

It is preferred that the addition polymerisation process comprises a free-radical polymerisation process and that the initiator comprises a free-radical initiator.

According to a third aspect of the present invention there is provided an oil-in-water or water-in-oil emulsion comprising an amphiphilic branched polymer according to the first aspect of the present invention. The amphiphilic branched polymer is preferably prepared by the method according to the second aspect of the present invention.

The emulsion may also further comprise a dispersed phase and an active ingredient may be incorporated in the dispersed phase. The average size of the droplets in the emulsion is less than 20 μm.

In the emulsion, the amphiphilic branched copolymer is incorporated into an oil and water mixture and thereby stabilises the oil-water interface. Whilst not wishing to be bound by any particular theory the stabilisation is preferably via dissolution of the polymer into one phase and homogenisation with the other phase. If the physical and chemical structure of the polymer is synthesised such that it is ‘tuned’ to respond to an external stimuli, when the stimuli is applied, the resultant changes to the polymer allow demulsification of the emulsion.

That is, the ‘tuning’ of the polymers allows for triggered demulsification of the emulsion.

According to a fourth aspect of the present invention there is described the use of the amphiphilic branched polymers according to the first aspect of the present invention as an emulsifier and/or as a triggered release agent.

Preferred amphiphilic branched copolymers according to the present invention include DEA₉₅/PEG_{1K}MA₅-PEGDMA₁₅-TG₁₇ and DEA₉₅/PEG_{1K}MA₅-PEGDMA₁₅-MPA₁₅ but are not limited thereto.

### Definitions

The following definitions pertain to chemical structures, molecular segments and substituents:

The ethylenically monounsaturated monomer is also referred to as ‘monofunctional monomer’. The ethylenically polyunsaturated monomer as ‘multifunctional monomer’.

The term ‘alkyl’ as used herein refers to a branched or un-branched saturated hydrocarbon group which may contain from 1 to 12 carbon atoms, such as methyl, ethyl, n-propyl, isopropyl, n-butyl, isobutyl, t-butyl, octyl, decyl and the like. Preferably, an alkyl group contains from 1 to 6, and more preferably from 1 to 4 carbon atoms. Methyl, ethyl and propyl groups are especially preferred. ‘Substituted alkyl’ refers to alkyl substituted with one or more substituent groups. Preferably, alkyl and substituted alkyl groups are un-branched.

Typical substituent groups include for example: halogen atoms, nitro, cyano, hydroxyl, cycloalkyl, alkyl, alkenyl, haloalkyl, alkoxy, haloalkoxy, amino, alkylamino, dialkylamino, formyl, alkoxycarbonyl, carboxyl, alkanoyl, alkylthio, alkylsulfinyl, alkylsulfonyl, alkylsulfonato, arylsulfinyl, arylsulfonyl, arylsulfonato, phosphinyl, phosphonyl, carbamoyl, amido, alkylamido, aryl, aralkyl and quaternary ammonium groups, such as betaine groups. Of these substituent groups, halogen atoms, cyano, hydroxyl, alkyl, haloalkyl, alkoxy, haloalkoxy, amino, carboxyl, amido and quaternary ammonium groups, or zwitterionic moieties such as betaine groups, are particularly preferred. When any of the foregoing substituents represents or contains an alkyl or alkenyl substituent group, this may be linear or branched and may contain up to 12, preferably up to 6, and especially up to 4, carbon atoms. A cycloalkyl group may contain from 3 to 12, preferably from 8 to 10, carbon atoms. An aryl group or moiety may contain from 6 to 10 carbon atoms, phenyl groups being especially preferred. A halogen atom may be a fluorine, chlorine, bromine or iodine atom and any group which contains a halo moiety, such as a haloalkyl group, may thus contain any one or more of these halogen atoms.

Terms such as ‘(meth)acrylic acid’ embrace both methacrylic acid and acrylic acid. Analogous terms should be construed similarly.

Molar percentages are based on the total monofunctional monomer content.

Molecular weights of monomers and polymers are expressed as weight average molecular weights (Mw), except where otherwise specified.

### DETAILED DESCRIPTION

Emulsions are important materials since they allow incompatible liquids to subsist within discrete domains as free-flowing dispersions and are used in diverse applications such as pharmaceuticals, agrochemicals, electronics, encapsulation and release of bio-important materials, gene therapy and transfer, foods, cosmetics, tertiary oil recovery and as templates for advanced materials fabrication. Responsive emulsions can be prepared if the surfactants or particles employed to stabilise the interface change their properties in response to external stimuli such as pH, temperature, gas or light. Furthermore, if the surfactant wettability at the droplet interface changes, these emulsions can demulsify on-demand. Interest in these systems is growing as release of large ‘pay-loads’ is desirable for many applications. However, the present invention predominantly relates to and is focussed on triggering rapid and complete demulsification.

Herein it is demonstrated that relatively subtle changes in branched copolymer architecture and chain-end can provide a significant degree of control over the properties of pH-responsive emulsions. For example, it is demonstrated herein the preparation of stable emulsion droplets wherein the surface functionality responds to solution pH whilst maintaining droplet integrity, and furthermore it is shown that rapid demulsification can be triggered. These differing behaviours are controlled by changing the length and hydrophilicity of the branching unit (architecture) and hydrophilicity of the chain-end thereby affecting the overall hydrophilic/lipophilic balance of the emulsifier and represent a significant development in the ability to tailor surfactant design for specific applications.

For a molecule to be surface active, and therefore act as a suitable and efficient emulsifier it must be amphiphilic in nature, that is contain hydrophilic or hydrophobic units. Upon formation of an emulsion these materials stabilise the oil/water interface resulting in the formation of a dispersed emulsion droplet in a continuous phase. Polymeric emulsifiers are particularly suited to this action as once positioned at the oil/water interface they are extremely difficult to remove due to their high molecular weight and inherent large physical size resulting in extremely stable emulsions when compared to their low molecular weight counterparts. Wholly insoluble particles, commonly described as Pickering or Ramsden emulsifiers, similarly give rise to stable emulsions. Block or comb polymers where a defined section of the polymeric unit is wholly hydrophilic or hydrophobic have been shown to be especially effective emulsifiers at low levels. There has also been recent interest in the literature concerning organic or organic/inorganic hybrid particulates whereby the outer shell of the particles have been tailored to interact with both emulsion phases resulting in strong anchoring at the oil/water interface resulting in stable and well defined emulsion droplets.

For a material to act as a responsive emulsifier it must fulfill the criteria described above whereby it can act as an efficient emulsifier. The material must then respond to a change in the emulsion's environment, chemical or physical, where it can demulsify or release the emulsion payload. For example the emulsifier may contain acidic or basic moieties which respond to the solution pH rendering the molecules charged whereby they repel each other from the oil/water interface resulting in demulsification or where the protonation/deprotonation of the groups within the molecule reduces the surface activity of the material again resulting in demulsification. The emulsifier could react to a thermal trigger whereby the material alters in conformation or solubility resulting in demulsification. The emulsifier could respond to an electromagnetic radiation trigger whereby the molecule changes confirmation or undergoes homolysis resulting in a decrease in surface activity, size or removal from the oil/water interface resulting in demulsification.

The branched polymer emulsifiers described in this present invention posses both hydrophilic and hydrophobic moieties within their structure resulting from the choice of monomer(s), brancher(s) and chain transfer agents(s). These units have been chosen such that their hydrophilic/hydrophobic natures can be altered through an external change. For the examples given, this change is an altering in solution pH. When a weakly basic monomer, such as 2-diethylaminoethyl methacrylate (DEA) is used as a monomer in the preparation of the branched polymer emulsifiers, the tertiary amine function will be hydrophilic or hydrophobic below or above the amine's pKa. Therefore when polymerised with a wholly hydrophilic monomer, such as poly(ethylene glycol) methacrylate (PEGMA) the overall surface activity and charge density of the polymer can be controlled through changes in the solution pH where at low pH values the molecule will be more hydrophilic and highly charged than at higher pH values where the DEA unit will be completely uncharged and the molecule will be more amphiphilic in nature.

By further optimising the overall hydrophilic/hydrophobic balance and the architecture of the weakly basic branched polymer emulsifiers their structures can be further tuned such that emulsions formed using these polymers can be triggered to demulsify via a decrease in solution pH. Wholly stable emulsions can also be prepared using branched polymers possessing hydrophobic end or side-groups, through the incorporation of alkyl monomers or chain transfer agents in the polymerisation, and or the utilisation of short-chain or hydrophobic branchers. It has been found that polymers of this type demulsify less or not at all upon external triggering due to their inherent amphiphilicity. Where the branched polymer emulsifier contains more hydrophilic groups, again via the use of hydrophilic monomers or chain transfer agents during the synthesis and or using longer chain hydrophilic branchers, such as a poly(ethylene oxide)-containing polyfunctional monomer, then the demulsification can be increased such that the emulsion can completely demulsify upon external triggering.

One measure of the emulsification efficiency is the extent to which the polymer solution lowers the surface tension of water, that is, how efficiently the polymer adsorbs at the air-water interface. This air-water interface may be used as a model to predict polymer adsorption at an oil-water interface. It is proposed that this is an accepted and suitable assumption since water is a constant in each system and both the air and oil phases are hydrophobic. Therefore it has been found that by measuring the air-water surface tension of different polymer solutions the efficiency of the polymers to act as emulsifiers can be derived. In the present invention it is thus shown that the air-water surface tensions correlate extremely well with emulsification efficiencies for the disclosed polymers. Since the polymers are pH-responsive (that is one of the monofunctional monomers changes its hydrophobicity in response to solution pH), the surface tension also changes in response to the solution pH. Consequently, by changing the solution pH, the emulsification efficiency also changes. If this change is sufficiently large, then the emulsions will demulsify (as the polymer will no longer adsorb at the oil-water interface).

It is also shown below:
i) the initial surface tension at a given solution pH to define the emulsification efficiency (that is, how suitable the polymers stabilise the emulsion droplets) and ii) the change in surface tension on changing the solution pH to define the amount of demulsification (that is, how well the polymers desorb from the oil-water interface and cause the emulsions to phase separate).

It is further shown that the initial emulsification efficiency is largely defined by the monofunctional monomers (that is, the type and ratio of the monomers) and that demulsification is largely defined by the chain-end and branching monomers.

The amphiphilic branched copolymers of the present invention are branched, non-cross-linked addition polymers and include statistical, block, graft, gradient and alternating branched copolymers. The copolymers of the present invention comprise at least two chains which are covalently linked by a bridge other than at their ends, that is, a sample of said copolymer comprises on average at least two chains which are covalently linked by a bridge other than at their ends. When a sample of the copolymer is made there may be accidentally some polymer molecules that are un-branched, which is inherent to the production method (addition polymerisation process). For the same reason, a small quantity of the polymer will not have a chain transfer agent (CTA) on the chain end.

The chain transfer agent (CTA) is a molecule which is known to reduce molecular weight during a free-radical polymerisation via a chain transfer mechanism. The amphiphilicity, emulsion stabilising power, responsive nature and susceptibility to controlled demulsification can be controlled through the choice of chain transfer agent. These agents may be any thiol-containing molecule and can be either monofunctional or multifunctional. The agent may be hydrophilic, hydrophobic, amphiphilic, anionic, cationic, neutral, zwitterionic or responsive. The molecule can also be an oligomer or a pre-formed polymer containing a thiol moiety. (The agent may also be a hindered alcohol or similar free-radical stabiliser). Catalytic chain transfer agents such as those based on transition metal complexes such as cobalt bis(borondifluorodimethyl-glyoximate) (CoBF) may also be used. Suitable thiols include but are not limited to C₂ to C₁₈ branched or linear alkyl thiols such as dodecane thiol, functional thiol compounds such as thioglycolic acid, thio propionic acid, thioglycerol, cysteine and cysteamine. Thiol-containing oligomers or polymers may also be used such as for example poly(cysteine) or an oligomer or polymer which has been post-functionalised to give a thiol group(s), such as poly(ethyleneglycol) (di)thio glycollate, or a pre-formed polymer functionalised with a thiol group. For example, the reaction of an end or side-functionalised alcohol such as poly(propylene glycol) with thiobutyrolactone, to give the corresponding thiol-functionalised chain-extended polymer. Multifunctional thiols may also be prepared by the reduction of a xanthate, dithioester or trithiocarbonate end-functionalised polymer prepared via a Reversible Addition Fragmentation Transfer (RAFT) or Macromolecular Design by the Interchange of Xanthates (MADIX) living radical method. Xanthates, dithioesters, and dithiocarbonates may also be used, such as cumyl phenyldithioacetate. Alternative chain transfer agents may be any species known to limit the molecular weight in a free-radical addition polymerisation including alkyl halides and transition metal salts or complexes. More than one chain transfer agent may be used in combination.

Hydrophobic CTAs include but are not limited to: linear and branched alkyl and aryl (di)thiols such as dodecanethiol, octadecyl mercaptan, 2-methyl-1-butanethiol and 1,9-nonanedithiol. Hydrophobic macro-CTAs (where the molecular weight of the CTA is at least 1000 Daltons) can be prepared from hydrophobic polymers synthesised by RAFT (or MADIX) followed by reduction of the chain end, or alternatively the terminal hydroxyl group of a preformed hydrophobic polymer can be post functionalised with a compound such as thiobutyrolactone.

Hydrophilic CTAs typically contain hydrogen bonding and/or permanent or transient charges. Hydrophilic CTAs include but are not limited to: thio-acids such as thioglycolic acid and cysteine, thioamines such as cysteamine and thio-alcohols such as 2-mercaptoethanol, thioglycerol and ethylene glycol mono- (and di-)thio glycollate. Hydrophilic macro-CTAs (where the molecular weight of the CTA is at least 1000 Daltons) can be prepared from hydrophilic polymers synthesised by RAFT (or MADIX) followed by reduction of the chain end, or alternatively the terminal hydroxyl group of a preformed hydrophilic polymer can be post functionalised with a compound such as thiobutyrolactone.

Amphiphilic CTAs can also be incorporated in the polymerisation mixture, these materials are typically hydrophobic alkyl-containing thiols possessing a hydrophilic function such as but not limited to a carboxylic acid group. Molecules of this type include mercapto undecylenic acid.

Responsive macro-CTAs (where the molecular weight of the CTA is at least 1000 Daltons) can be prepared from responsive polymers synthesised by RAFT (or MADIX) followed by reduction of the chain end, or alternatively the terminal hydroxyl group of a preformed responsive polymer, such as poly(propylene glycol), can be post functionalised with a compound such as thiobutyrolactone.

The residue of the chain transfer agent may comprise 0 to 80 mole % of the copolymer (based on the number of moles of monofunctional monomer). More preferably the residue of the chain transfer agent comprises 0 to 50 mole %, even more preferably 0 to 40 mole % of the copolymer (based on the number of moles of monofunctional monomer). However, most especially the chain transfer agent comprises 0.05 to 30 mole %, of the copolymer (based on the number of moles of monofunctional monomer).

The initiator is a free-radical initiator and can be any molecule known to initiate free-radical polymerisation such as for example azo-containing molecules, persulfates, redox initiators, peroxides, benzyl ketones. These may be activated via thermal, photolytic or chemical means. Examples of these include but are not limited to: 2,2′-azobisisobutyronitrile (AIBN), azobis(4-cyanovaleric acid), benzoyl peroxide, diisopropyl peroxide, cumylperoxide, 1-hydroxycyclohexyl phenyl ketone, hydrogenperoxide/ascorbic acid. Iniferters such as benzyl-N,N-diethyldithiocarbamate can also be used. In some cases, more than one initiator may be used. The initiator may be a macroinitiator having a molecular weight of at least 1000 Daltons. In this case, the macroinitiator may be hydrophilic, hydrophobic, or responsive. The amphiphilicity, emulsion stabilising power, responsive nature and susceptibility to controlled demulsification can be controlled through the choice of initiator, especially in the case where macromolecular pseudo living radical initiators are utilised.

Preferably, the residue of the initiator in a free-radical polymerisation comprises 0 to 5% w/w of the copolymer based on the total weight of the monomers. More preferably 0.01 to 5% w/w of the copolymer, and especially 0.01 to 3% w/w, of the copolymer based on the total weight of the monomers.

The use of a chain transfer agent and an initiator is preferred. However, some molecules can perform both functions.

Hydrophilic macroinitiators (where the molecular weight of the preformed polymer is at least 1000 Daltons) can be prepared from hydrophilic polymers synthesised by RAFT (or MADIX), or where a functional group of a preformed hydrophilic polymer, such as terminal hydroxyl group, can be post-functionalised with a functional halide compound, such as 2-bromoisobutyryl bromide, for use in Atom Transfer Radical Polymerisation (ATRP) with a suitable low valency transition metal catalyst, such as CuBr Bipyridyl.

Hydrophobic macroinitiators (where the molecular weight of the preformed polymer is at least 1000 Daltons) can be prepared from hydrophobic polymers synthesised by RAFT (or MADIX), or where a functional group of a preformed hydrophilic polymer, such as terminal hydroxyl group, can be post-functionalised with a functional halide compound, such as 2-bromoisobutyryl bromide, for use in Atom Transfer Radical Polymerisation (ATRP) with a suitable low valency transition metal catalyst, such as CuBr Bipyridyl.

Responsive macroinitiators (where the molecular weight of the preformed polymer is at least 1000 Daltons) can be prepared from responsive polymers synthesised by RAFT (or MADIX), or where a functional group of a preformed hydrophilic polymer, such as terminal hydroxyl group, can be post-functionalised with a functional halide compound, such as 2-bromoisobutyryl bromide, for use in Atom Transfer Radical Polymerisation (ATRP) with a suitable low valency transition metal catalyst, such as CuBr Bipyridyl.

The monofunctional monomer may comprise any carbon-carbon unsaturated compound which can be polymerised by an addition polymerisation mechanism, for example vinyl and allyl compounds. The amphiphilicity, emulsion stabilising power, responsive nature and susceptibility to controlled demulsification can be controlled through the choice of monofunctional monomer. The monofunctional monomer may be hydrophilic, hydrophobic, amphiphilic, anionic, cationic, neutral or zwitterionic in nature. The monofunctional monomer may be selected from but not limited to monomers such as:

vinyl acids, vinyl acid esters, vinyl aryl compounds, vinyl acid anhydrides, vinyl amides, vinyl ethers, vinyl amines, vinyl aryl amines, vinyl nitriles, vinyl ketones, and derivatives of the aforementioned compounds as well as corresponding allyl variants thereof

Other suitable monofunctional monomers include: hydroxyl-containing monomers and monomers which can be post-reacted to form hydroxyl groups, acid-containing or acid-functional monomers, zwitterionic monomers and quaternised amino monomers. Oligomeric, polymeric and di- or multi-functionalised monomers may also be used, especially oligomeric or polymeric (meth)acrylic acid esters such as mono(alk/aryl) (meth)acrylic acid esters of polyalkyleneglycol or polydimethylsiloxane or any other mono-vinyl or allyl adduct of a low molecular weight oligomer. Mixtures of more than one monomer may also be used to give statistical, graft, gradient or alternating copolymers.

In some cases, the monofunctional monomer are vinylic or allylic and are selected from the group consisting of styrenics, acrylics, methacrylics, allylics, acrylamides, methacrylamides, vinyl acetates, allyl acetates, N-vinyl amines, allyl amines, vinyl ethers, and allyl ethers.

Vinyl acids and derivatives thereof include: (meth)acrylic acid, fumaric acid, maleic acid, itaconic acid and acid halides thereof such as (meth)acryloyl chloride. Vinyl acid esters and derivatives thereof include: C₁ to C₂₀ alkyl(meth)acrylates (linear & branched) such as for example methyl (meth)acrylate, stearyl (meth)acrylate and 2-ethyl hexyl (meth)acrylate; aryl(meth)acrylates such as for example benzyl (meth)acrylate; tri(alkyloxy)silylalkyl(meth)acrylates such as trimethoxysilylpropyl(meth)acrylate; and activated esters of (meth)acrylic acid such as N-hydroxysuccinamido (meth)acrylate. Vinyl aryl compounds and derivatives thereof include: styrene, acetoxystyrene, styrene sulfonic acid, vinyl pyridine, vinylbenzyl chloride and vinyl benzoic acid. Vinyl acid anhydrides and derivatives thereof include: maleic anhydride. Vinyl amides and derivatives thereof include: (meth)acrylamide, N-(2-hydroxypropyl)methacrylamide, N-vinyl pyrrolidone, N-vinyl formamide, (meth)acrylamidopropyl trimethyl ammonium chloride, [3-((meth)acrylamido)propyl]dimethyl ammonium chloride, 3-[N-(3-(meth)acrylamidopropyl)-N,N-dimethyl]aminopropane sulfonate, methyl (meth)acrylamidoglycolate methyl ether and N-isopropyl(meth)acrylamide. Vinyl ethers and derivatives thereof include: methyl vinyl ether. Vinyl amines and derivatives thereof include: dimethylaminoethyl (meth)acrylate, diethylaminoethyl (meth)acrylate, diisopropylaminoethyl (meth)acrylate, mono-t-butylaminoethyl(meth)acrylate, morpholinoethyl(meth)acrylate and monomers which can be post-reacted to form amine groups, such as vinyl formamide. Vinyl aryl amines and derivatives thereof include: vinyl aniline, vinyl pyridine, N-vinyl carbazole and vinyl imidazole. Vinyl nitriles and derivatives thereof include: (meth)acrylonitrile. Vinyl ketones and derivatives thereof include: acreolin.

Hydroxyl-containing monomers include: vinyl hydroxyl monomers such as hydroxyethyl (meth)acrylate, hydroxy propyl (meth)acrylate, glycerol mono(meth)acrylate and sugar mono(meth)acrylates such as glucose mono(meth)acrylate. Monomers which can be post-reacted to form hydroxyl groups include: vinyl acetate, acetoxystyrene and glycidyl (meth)acrylate. Acid-containing or acid functional monomers include: (meth)acrylic acid, styrene sulfonic acid, vinyl phosphonic acid, vinyl benzoic acid, maleic acid, fumaric acid, itaconic acid, 2-(meth)acrylamido 2-ethyl propanesulfonic acid, mono-2-((meth)acryloyloxy)ethyl succinate and ammonium sulfatoethyl (meth)acrylate. Zwitterionic monomers include: (meth)acryloyl oxyethylphosphoryl choline and betaines, such as [2-((meth)acryloyloxy)ethyl] dimethyl-(3-sulfopropyl)ammonium hydroxide. Quaternised amino monomers include: (meth)acryloyloxyethyltri-(alk/aryl)ammonium halides such as (meth)acryloyloxyethyltrimethyl ammonium chloride.

Oligomeric and polymeric monomers include: oligomeric and polymeric (meth)acrylic acid esters such as mono(alk/aryl)oxypolyalkyleneglycol(meth)acrylates and mono(alk/aryl)oxypolydimethyl-siloxane(meth)acrylates. These esters include for example: monomethoxy oligo(ethyleneglycol) mono(meth)acrylate, monomethoxy oligo(propyleneglycol) mono(meth)acrylate, monohydroxy oligo(ethyleneglycol) mono(meth)acrylate, monohydroxy oligo (propylene glycol) mono(meth)acrylate, monomethoxy poly(ethyleneglycol) mono(meth)acrylate, monomethoxy poly(propyleneglycol) mono(meth)acrylate, monohydroxy poly(ethyleneglycol) mono(meth)acrylate and monohydroxy poly(propyleneglycol) mono(meth)acrylate. Further examples include: vinyl or allyl esters, amides or ethers of pre-formed oligomers or polymers formed via ring-opening polymerisation such as oligo(caprolactam), oligo(caprolactone), poly(caprolactam) or poly(caprolactone), or oligomers or polymers formed via a living polymerisation technique such as poly(1,4-butadiene).

The corresponding allyl monomers to those listed above can also be used where appropriate.

Examples of monofunctional monomers are: amide-containing monomers such as (meth)acrylamide, N-(2-hydroxypropyl)methacrylamide, N,N′-dimethyl(meth)acrylamide, N and/or N′-di(alkyl or aryl) (meth)acrylamide, N-vinyl pyrrolidone, [3-((meth)acrylamido)propyl] trimethyl ammonium chloride, 3-(dimethylamino)propyl(meth)acrylamide, 3-[N-(3-(meth)acrylamidopropyl)-N,N-dimethyl]aminopropane sulfonate, methyl (meth)acrylamidoglycolate methyl ether and N-isopropyl(meth)acrylamide; (Meth)acrylic acid and derivatives thereof such as (meth)acrylic acid, (meth)acryloyl chloride (or any halide), (alkyl/aryl)(meth)acrylate; functionalised oligomeric or polymeric monomers such as monomethoxy oligo(ethyleneglycol) mono(meth)acrylate, monomethoxy oligo(propyleneglycol) mono(meth)acrylate, monohydroxy oligo(ethyleneglycol) mono(meth)acrylate, monohydroxy oligo(propyleneglycol) mono(meth)acrylate.monomethoxy poly(ethyleneglycol) mono(meth)acrylate, monomethoxy poly(propyleneglycol) mono(meth)acrylate, monohydroxy poly(ethyleneglycol) mono(meth)acrylate, monohydroxy poly(propyleneglycol) mono(meth)acrylate. glycerol mono(meth)acrylate and sugar mono(meth)acrylates such as glucose mono(meth)acrylate; vinyl amines such as aminoethyl (meth)acrylate, dimethylaminoethyl (meth)acrylate, diethylaminoethyl (meth)acrylate, diisopropylaminoethyl (meth)acrylate, mono-t-butylamino (meth)acrylate, morpholinoethyl(meth)acrylate; vinyl aryl amines such as vinyl aniline, vinyl pyridine, N-vinyl carbazole, vinyl imidazole, and monomers which can be post-reacted to form amine groups, such as vinyl formamide; vinyl aryl monomers such as styrene, vinyl benzyl chloride, vinyl toluene, a-methyl styrene, styrene sulfonic acid and vinyl benzoic acid; vinyl hydroxyl monomers such as hydroxyethyl (meth)acrylate, hydroxy propyl (meth)acrylate, glycerol mono(meth)acrylate or monomers which can be post-functionalised into hydroxyl groups such as vinyl acetate, acetoxy styrene and glycidyl (meth)acrylate; acid-containing monomers such as (meth)acrylic acid, styrene sulfonic acid, vinyl phosphonic acid, vinyl benzoic acid, maleic acid, fumaric acid, itaconic acid, 2-(meth)acrylamido 2-ethyl propanesulfonic acid and mono-2-((meth)acryloyloxy)ethyl succinate or acid anhydrides such as maleic anhydride; zwitterionic monomers such as (meth)acryloyl oxyethylphosphoryl choline and betaine-containing monomers, such as [2-((meth)acryloyloxy)ethyl] dimethyl-(3-sulfopropyl)ammonium hydroxide; quaternised amino monomers such as (meth)acryloyloxyethyltrimethyl ammonium chloride.

The corresponding allyl monomer, where applicable, can also be used in each case.

Functional monomers, that is monomers with reactive pendant groups which can be post or pre-modified with another moiety following polymerisation can also be used such as for example glycidyl (meth)acrylate, tri(alkoxy)silylalkyl(meth)acrylates such as trimethoxysilylpropyl(meth)acrylate, (meth)acryloyl chloride, maleic anhydride, hydroxyalkyl (meth)acrylates, (meth)acrylic acid, vinylbenzyl chloride, activated esters of (meth)acrylic acid such as N-hydroxysuccinamido (meth)acrylate and acetoxystyrene.

Macromonomers (monomers having a molecular weight of at least 1000 Daltons) are generally formed by linking a polymerisable moiety, such as a vinyl or allyl group, to a pre-formed monofunctional polymer via a suitable linking unit such as an ester, an amide or an ether. Examples of suitable polymers include: mono functional poly(alkylene oxides) such as monomethoxy[poly(ethyleneglycol)] or monomethoxy[poly(propyleneglycol)], silicones such as poly(dimethylsiloxane)s, polymers formed by ring-opening polymerisation such as poly(caprolactone) or poly(caprolactam) or mono-functional polymers formed via living polymerisation such as poly(1,4-butadiene).

Preferred macromonomers include: monomethoxy[poly(ethyleneglycol)] mono(methacrylate), monomethoxy[poly(propyleneglycol)] mono(methacrylate) and mono(meth)acryloxypropyl-terminated poly(dimethylsiloxane).

When the monofunctional monomer is providing the necessary hydrophilicity in the copolymer, it is preferred that the monofunctional monomer is a residue of a hydrophilic monofunctional monomer, preferably having a molecular weight of at least 1000 Daltons.

Hydrophilic monofunctional monomers include: (meth)acryloyl chloride, N-hydroxysuccinamido (meth)acrylate, styrene sulfonic acid, maleic anhydride, (meth)acrylamide, N-(2-hydroxypropyl)methacrylamide, N-vinyl pyrrolidinone, N-vinyl formamide, quaternised amino monomers such as (meth)acrylamidopropyl trimethyl ammonium chloride, [3-((meth)acrylamido)propyl]trimethyl ammonium chloride and (meth)acryloyloxyethyltrimethyl ammonium chloride, 3-[N-(3-(meth)acrylamidopropyl)-N,N-dimethyl]aminopropane sulfonate, methyl (meth)acrylamidoglycolate methyl ether, glycerol mono(meth)acrylate, monomethoxy and monohydroxy oligo(ethylene oxide) (meth)acrylate, sugar mono(meth)acrylates such as glucose mono(meth)acrylate, (meth)acrylic acid, vinyl phosphonic acid, fumaric acid, itaconic acid, 2-(meth)acrylamido 2-ethyl propanesulfonic acid, mono-2-((meth)acryloyloxy)ethyl succinate, ammonium sulfatoethyl (meth)acrylate, (meth)acryloyl oxyethylphosphoryl choline and betaine-containing monomers such as [2-((meth)acryloyloxy)ethyl] dimethyl-(3-sulfopropyl)ammonium hydroxide. Hydrophilic macromonomers may also be used and include: monomethoxy and monohydroxy poly(ethylene oxide) (meth)acrylate and other hydrophilic polymers with terminal functional groups which can be post-functionalised with a polymerisable moiety such as (meth)acrylate, (meth)acrylamide or styrenic groups.

Hydrophobic monofunctional monomers include: C₁ to C₂₀ alkyl (meth)acrylates (linear and branched and (meth)acrylamides, such as methyl (meth)acrylate and stearyl (meth)acrylate, aryl(meth)acrylates such as benzyl (meth)acrylate, tri(alkyloxy)silylalkyl(meth)acrylates such as trimethoxysilylpropyl(meth)acrylate, styrene, acetoxystyrene, vinylbenzyl chloride, methyl vinyl ether, vinyl formamide, (meth)acrylonitrile, acreolin, 1- and 2-hydroxy propyl (meth)acrylate, vinyl acetate, and glycidyl (meth)acrylate. Hydrophobic macromonomers may also be used and include: monomethoxy and monohydroxy poly(butylene oxide) (meth)acrylate and other hydrophobic polymers with terminal functional groups which can be post-functionalised with a polymerisable moiety such as (meth)acrylate, (meth)acrylamide or styrenic groups.

Responsive monofunctional monomers include: (meth)acrylic acid, 2- and 4-vinyl pyridine, vinyl benzoic acid, N-isopropyl(meth)acrylamide, tertiary amine (meth)acrylates and (meth)acrylamides such as 2-(dimethyl)aminoethyl (meth)acrylate, 2-(diethylamino)ethyl (meth)acrylate, diisopropylaminoethyl (meth)acrylate, mono-t-butylaminoethyl (meth)acrylate and N-morpholinoethyl (meth)acrylate, vinyl aniline, vinyl pyridine, N-vinyl carbazole, vinyl imidazole, hydroxyethyl (meth)acrylate, hydroxypropyl (meth)acrylate, maleic acid, fumaric acid, itaconic acid and vinyl benzoic acid. Responsive macromonomers may also be used and include: monomethoxy and monohydroxy poly(propylene oxide) (meth)acrylate and other responsive polymers with terminal functional groups which can be post-functionalised with a polymerisable moiety such as (meth)acrylate, (meth)acrylamide or styrenic groups.

The multifunctional monomer or brancher may comprise a molecule containing at least two vinyl groups which may be polymerised via addition polymerisation. The molecule may be hydrophilic, hydrophobic, amphiphilic, neutral, cationic, zwitterionic, oligomeric or polymeric. Such molecules are often known as cross-linking agents in the art and may be prepared by reacting any di- or multifunctional molecule with a suitably reactive monomer. Examples include: di- or multivinyl esters, di- or multivinyl amides, di- or multivinyl aryl compounds, di- or multivinyl alk/aryl ethers. Typically, in the case of oligomeric or polymeric di- or multifunctional branching agents, a linking reaction is used to attach a polymerisable moiety to a di- or multifunctional oligomer or polymer. The brancher may itself have more than one branching point, such as T-shaped divinylic oligomers or polymers. In some cases, more than one multifunctional monomer may be used. When the multifunctional monomer is providing the necessary hydrophilicity in the copolymer, it is preferred that the multifunctional monomer has a molecular weight of at least 1000 Daltons.

The corresponding allyl monomers to those listed above can also be used where appropriate.

Preferred multifunctional monomers include but are not limited to divinyl aryl monomers such as divinyl benzene; (meth)acrylate diesters such as ethylene glycol di(meth)acrylate, propyleneglycol di(meth)acrylate and 1,3-butylenedi(meth)acrylate; polyalkylene oxide di(meth)acrylates such as tetraethyleneglycol di(meth)acrylate, poly(ethyleneglycol) di(meth)acrylate and poly(propyleneglycol) di(meth)acrylate; divinyl (meth)acrylamides such as methylene bisacrylamide; silicone-containing divinyl esters or amides such as (meth)acryloxypropyl-terminated poly(dimethylsiloxane); divinyl ethers such as poly(ethyleneglycol)divinyl ether; and tetra- or tri-(meth)acrylate esters such as pentaerythritol tetra(meth)acrylate, trimethylolpropane tri(meth)acrylate or glucose di- to penta(meth)acrylate. Further examples include vinyl or allyl esters, amides or ethers of pre-formed oligomers or polymers formed via ring-opening polymerisation such as oligo(caprolactam), oligo(caprolactone), poly(caprolactam) or poly(caprolactone), or oligomers or polymers formed via a living polymerisation technique such as oligo- or poly(1,4-butadiene).

Macrocrosslinkers or macrobranchers (multifunctional monomers having a molecular weight of at least 1000 Daltons) are generally formed by linking a polymerisable moiety, such as a vinyl or aryl group, to a pre-formed multifunctional polymer via a suitable linking unit such as an ester, an amide or an ether. Examples of suitable polymers include: di-functional poly(alkylene oxides) such as poly(ethyleneglycol) or poly(propyleneglycol), silicones such as poly(dimethylsiloxane)s, polymers formed by ring-opening polymerisation such as poly(caprolactone) or poly(caprolactam) or poly-functional polymers formed via living polymerisation such as poly(1,4-butadiene).

Preferred macrobranchers include: poly(ethyleneglycol) di(meth)acrylate, poly(propyleneglycol) di(meth)acrylate, methacryloxypropyl-terminated poly(dimethylsiloxane), poly(caprolactone) di(meth)acrylate and poly(caprolactam) di(meth)acrylamide.

Branchers include: methylene bisacrylamide, glycerol di(meth)acrylate, glucose di- and tri(meth)acrylate, oligo(caprolactam) and oligo(caprolactone). Multi end-functionalised hydrophilic polymers may also be functionalised using a suitable polymerisable moiety such as a (meth)acrylate, (meth)acrylamide or styrenic group.

Further branchers include: divinyl benzene, (meth)acrylate esters such as ethyleneglycol di(meth)acrylate, propyleneglycol di(meth)acrylate and 1,3-butylene di(meth)acrylate, oligo(ethylene glycol) di(meth)acrylates such as tetraethylene glycol di(meth)acrylate, tetra- or tri-(meth)acrylate esters such as pentaerthyritol tetra(meth)acrylate, trimethylolpropane tri(meth)acrylate and glucose penta(meth)acrylate. Multi end-functionalised hydrophobic polymers may also be functionalised using a suitable polymerisable moiety such as a (meth)acrylate, (meth)acrylamide or styrenic group.

Multifunctional responsive polymers may also be functionalised using a suitable polymerisable moiety such as a (meth)acrylate, (meth)acrylamide or styrenic group such as poly(propylene oxide) di(meth)acrylate.

Synthetic method. As highlighted above according to the second aspect of the present invention there is provided a method of preparing an amphiphilic branched copolymer according to any one of claims 1 to 4 by an addition polymerisation process, which comprises the mixing together of:
i) at least one ethylenically monounsaturated monomer;
ii) from 1 to 25 mole % (based on the number of moles of monofunctional monomer(s)) of at least one ethylenically polyunsaturated monomer;
iii) a chain transfer agent; and
iv) an initiator, and subsequently reacting said mixture to form a branched copolymer.

The copolymer is preferably prepared by an addition polymerisation method, which is a conventional free-radical polymerisation technique using a chain transfer agent.

To produce a branched polymer by a conventional radical polymerisation process, a monofunctional monomer is polymerised with a multifunctional monomer or branching agent in the presence of a chain transfer agent and free-radical initiator.

The polymerisations may proceed via solution, bulk, suspension, dispersion or emulsion procedures.

According to the third aspect of the present invention there is provided an oil-in-water or water-in-oil emulsion comprising an amphiphilic branched polymer according to the first aspect of the present invention and/or prepared by the method according to the second aspect of the present invention wherein the polymer is located at the oil-water interface.

Preferably the average size of the droplets in the emulsion is less than 20 nm, more preferably less than 10 μm. It is also preferred that the emulsion is an oil-in-water emulsion.

In a preferred embodiment the emulsion comprises an active ingredient, and the active ingredient is incorporated in the dispersed phase.

In a fourth aspect of the present invention there is provided the use of the designed and prepared responsive amphiphilic branched polymer as an emulsifier to provide an emulsion that may be triggered to demulsify upon the application of an external stimuli. Such polymers will therefore exist as stable emulsions until such time as a trigger or stimulus is actuated.

In a preferred embodiment the emulsion will completely demulsify releasing the dispersed phase and any active ingredient into the bulk phase.

The demulsification step will be preferably triggered by a physical or chemical change such as for example pH, temperature, electromagnetic radiation, ionic strength, change in concentration, addition of a further chemical entity or by sonic means.

### EXAMPLES

The present invention will now be explained in more detail by reference to the following non-limiting examples.

In the following examples, copolymers are described using the following nomenclature: (MonomerG)_{g} (Monomer J)ⱼ (Brancher L)ₗ(Chain Transfer Agent)_{d} wherein the values in subscript are the molar ratios of each constituent normalised to give the monofunctional monomer values as 100, that is, g+j=100. The degree of branching or branching level is denoted by l and d refers to the molar ratio of the chain transfer agent.

For example: Methacrylic acid₁₀₀ Ethyleneglycol dimethacrylate₁₅ Dodecane thiol₁₅ would describe a polymer containing methacrylic acid:ethyleneglycol dimethacrylate:dodecane thiol at a molar ratio of 100:15:15.

According to the present invention six polymers were synthesised to identify the effect of chain-end and internal architecture (for example long, poly(ethyleneglycol) dimethacrylate (PEGDMA), or short, ethyleneglycol dimethacrylate (EGDMA) branching monomers) on emulsion behaviour.

Branched copolymers were synthesised. PEGMA, DEA, branching monomer (either EGDMA or PEGDMA) and chain transfer agent where added to a glass vessel equipped with stirrer bar in pre-determined molar ratios (represented as subscripts in Table 1) and degassed by nitrogen purge for 30 minutes. Ethanol was degassed separately and added to the monomer mixture to give a 10% solution which was heated to 70° C. under an inert atmosphere. Polymerization was started by addition of 2,2′azo-bis-isobutyronitrile (AIBN) and the reaction was left stirring for 48 hours. After this time monomer conversions in excess of 97% were typically achieved and ethanol was removed by evaporation at reduced pressure. Any unreacted components were removed by precipitation of the polymer into cold diethyl ether or n-hexane. The resulting materials were dried in a vacuum oven over night and then characterized. ¹H NMR of the purified polymers (CDCl₃) revealed that the average molar ratios of the PEGMA to DEA were accurate to within 15% in all cases when compared to the target polymer composition when these proton resonances could be deconvoluted (that is, examples 1 to 3).

Emulsions were prepared by homogenization of aqueous polymer solutions (0.5%) at pH 10 with an equal volume of n-dodecane oil using an IKA Ultra-Turrax T25 homogenizer at 24,000 rpm for 2 minutes. Emulsions were left for at least 24 hours to equilibrate before characterization.

Demulsification of the branched copolymer-stabilized emulsions was triggered by addition of HCl (1M, 100 μL) to the creamed emulsion after 24 hours equilibration (1 mL). The extent of demulsification was determined visually by measuring the volume of oil which separated (as a clear top phase) from the emulsion as a function of time.

Emulsions were analyzed by light microscopy and laser diffraction. For light microscopy, a drop of the emulsion was placed on a glass slide and viewed using a Meiji Techno MX9000 Series microscope equipped with a Luminera Infinity 1 digital camera. Emulsion droplet diameters and diameter distributions were measured using a Malvern Mastersizer 2000 equipped with a Hydro 2000 SM dispersion unit. A drop of the emulsion was added to the dispersion unit containing approximately 100 mL water (adjusted to either pH 10 using sodium hydroxide (NaOH) or pH 2 using hydrochloric acid (HC1)) with a stirring rate of 1000 rpm. The Mastersizer cell was repeatedly rinsed using basic water and ethanol after each run. The volume-average droplet diameters (D_{4/3}) quoted were obtained from at least 20 repeat runs (D_{4/3}=ΣDᵢ⁴Nᵢ/ΣDᵢ³Nᵢ).

Polymer 1 comprises a short chain brancher and hydrophobic chain-end.

Polymer 2 comprises a short chain brancher and hydrophilic (neutral) chain-end.

Polymer 3 comprises a short chain brancher and hydrophilic (anionic) chain-end.

Polymer 4 comprises a long chain brancher and hydrophobic chain-end.

Polymer 5 comprises a long chain brancher and hydrophilic (neutral) chain-end.

Polymer 6 comprises a long chain brancher and hydrophilic (anionic) chain-end.

### 1) DEA₉₅/PEG_{1K}MA₅-EGDMA₁₅-DDT₁₅

PEGMA (4.000 g, 3.64 mM), DEA (12.782 g, 69.1 mM), EGDMA (2.160 g, 10.9 mM) and DDT (2.200 g, 10.9 mM) where added to a glass vessel equipped with stirrer bar and degassed by nitrogen purge for 30 minutes. Ethanol was degassed separately and added to the monomer mixture (211 mL) which was heated to 70° C. under an inert atmosphere. Polymerization was started by addition of AIBN (211 mg) and the reaction was left stirring for 48 hours. After this time ethanol was removed by evaporation at reduced pressure. The polymer was washed with cold diethyl ether and n-hexane and dried in a vacuum oven over night.

### 2) DEA₉₅/PEG_{1K}MA₅-EGDMA₁₅-TG₁₇

PEGMA (4.000 g, 3.64 mM), DEA (12.782 g, 69.1 mM), EGDMA (2.160 g, 10.9 mM) and TG (0.612 g, 12.4 mM) where added to a glass vessel equipped with stirrer bar and degassed by nitrogen purge for 30 minutes. Ethanol was degassed separately and added to the monomer mixture (211 mL) which was heated to 70° C. under an inert atmosphere. Polymerization was started by addition of AIBN (211 mg) and the reaction was left stirring for 48 hours. After this time ethanol was removed by evaporation at reduced pressure. The polymer was washed with cold diethyl ether and n-hexane and dried in a vacuum oven over night.

### 3) DEA₉₅/PEG_{1K}MA₅-EGDMA₁₅-MPA₁₅

PEGMA (1.878 g, 1.7 mM), DEA (6.000 g, 32 mM), EGDMA (1.014 g, 5 mM) and MPA (0.543 g, 5 mM) were added to a glass vessel equipped with stirrer bar and degassed by nitrogen purge for 30 minutes. Ethanol was degassed separately and added to the monomer mixture (90 mL) which was heated to 70° C. under an inert atmosphere. Polymerization was started by addition of AIBN (90 mg) and the reaction was left stirring for 48 hours. After this time ethanol was removed by evaporation at reduced pressure. The polymer was washed with cold diethyl ether and n-hexane and dried in a vacuum oven over night.

### 4) DEA₉₅/PEG_{1K}MA₅-PEGDMA₁₅-DDT₁₅

PEGMA (1.878 g, 1.7 mM), DEA (6.000 g, 32 mM), PEGDMA (4.375 g, 5 mM) and DDT (1.01 g, 5 mM) were added to a glass vessel equipped with stirrer bar and degassed by nitrogen purge for 30 minutes. Ethanol was degassed separately and added to the monomer mixture (90 mL) to give a 10% solution which was heated to 70° C. under an inert atmosphere. Polymerization was started by addition of AIBN (90 mg) and the reaction was left stirring for 48 hours. After this time ethanol was removed by evaporation at reduced pressure. The polymer was washed with cold diethyl ether and n-hexane and dried in a vacuum oven over night.

### 5) DEA₉₅/PEG_{1K}MA₅-PEGDMA₁₅-TG₁₇

PEGMA (1.878 g, 1.7 mM), DEA (6.000 g, 32 mM), PEGDMA (4.375 g, 5 mM) and TG (0.624 g, 5.8 mM) were added to a glass vessel equipped with stirrer bar and degassed by nitrogen purge for 30 minutes. Ethanol was degassed separately and added to the monomer mixture (90 mL) which was heated to 70° C. under an inert atmosphere. Polymerization was started by addition of AIBN (90 mg) and the reaction was left stirring for 48 hours. After this time ethanol was removed by evaporation at reduced pressure. The polymer was washed with cold diethyl ether and n-hexane and dried in a vacuum oven over night.

### 6) DEA₉₅/PEG_{1K}MA₅-PEGDMA₁₅-MPA₁₅

PEGMA (1.878 g, 1.7 mM), DEA (6.000 g, 32 mM), PEGDMA (4.375 g, 5 mM) and MPA (0.530 g, 5 mM) were added to a glass vessel equipped with stirrer bar and degassed by nitrogen purge for 30 minutes. Ethanol was degassed separately and added to the monomer mixture (90 mL) which was heated to 70° C. under an inert atmosphere. Polymerization was started by addition of AIBN (90 mg) and the reaction was left stirring for 48 hours. After this time ethanol was removed by evaporation at reduced pressure. The polymer was washed with cold diethyl ether and n-hexane and dried in a vacuum oven over night.

### Demulsification Data:

| | | Branched Copolymer | | | | | | |
|---|---|---|---|---|---|---|---|---|
| | | Characterization | | | | | | |
| | | | | | | Surface | Emulsion Characterization | |
| | Target Polymer | Mₙ/ | | MH- | Dₕ/ | tension/ | D_{(4,3)}/ | Percentage |
| Sample | Composition | g.mol^{-1a} | PDI ^{a} | α ^{a} | nm ^{b} | mN/m ^{c} | μm ^{d} | demulsification ^{e} |
| 1 | PEGMA₅/DEA₉₅- | 7,400 | 2.8 | 0.31 | 22 | 41.7 | 8.7 | 0 |
| | EGDMA₁₅-DDT₁₅ | | | | | | | |
| 2 | PEGMA₅/DEA₉₅- | 20,500 | 2.8 | 0.30 | 26 | 45.4 | 10.6 | 30 |
| | EGDMA₁₅-TG₁₇ | | | | | | | |
| 3 | PEGMA₅/DEA₉₅- | 8,500 | 3.3 | 0.31 | 13 | 50.7 | 8.4 | 50 |
| | EGDMA₁₅-MPA₁₅ | | | | | | | |
| 4 | PEGMA₅/DEA₉₅- | 9,000 | 4.5 | 0.39 | 11 | 40.4 | 11.6 | <1 |
| | PEGDMA₁₅-DDT₁₅ | | | | | | | |
| 5 | PEGMA₅/DEA₉₅- | 25,800 | 7.9 | 0.44 | 24 | 55.5 | 12.5 | >99 |
| | PEGDMA₁₅-TG₁₇ | | | | | | | |
| 6 | PEGMA₅/DEA₉₅- | 18,900 | 6.2 | 0.39 | 21 | 57.8 | 10.4 | >99 |
| | PEGDMA₁₅-MPA₁₅ | | | | | | | |

^{a} Measured by triple-detection THF GPC;
^{b} Measured by dynamic light scattering for a 0.5% aqueous polymer solution at pH 10;
^{c} 1.0% polymer solution at pH 2;
^{d} Measured by laser diffraction at pH 10;
^{e} Quantified by measuring oil volume separated 12 hours after addition of acid.

In conclusion, polymer surfactant architecture and chain-end functionality can significantly affect the behavior of responsive copolymer stabilized emulsions. Stable emulsions can be prepared where surface charge changes with variation of solution pH if hydrophobic chain-ends are employed. If long-chain branching units and hydrophilic chain-ends are employed, these polymers have sufficient chain mobility to efficiently de-wet from the droplet surfaces thus inducing demulsification. Short-chain branching units appear to restrict the ‘responsiveness’ of the branched copolymers in terms of inhibiting demulsification and this finding could have much broader implications in other fields where responsive polymeric materials are exploited.

Various dimensions, sizes, quantities, volumes, rates, and other numerical parameters and numbers have been used for purposes of illustration and exemplification of the principles of the invention, and are not intended to limit the invention to the numerical parameters and numbers illustrated, described or otherwise stated herein. Likewise, unless specifically stated, the order of steps is not considered critical. The different teachings of the embodiments discussed below may be employed separately or in any suitable combination to produce desired results.

While preferred embodiments of the invention have been shown and described, modifications thereof can be made by one skilled in the art without departing from the spirit and teachings of the invention. The embodiments described herein are exemplary only, and are not intended to be limiting. Many variations and modifications of the invention disclosed herein are possible and are within the scope of the invention. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, and so forth). Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, and the like.

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the preferred embodiments of the present invention. The disclosures of all patents, patent applications, and publications cited herein are hereby incorporated by reference, to the extent they provide exemplary, procedural or other details supplementary to those set forth herein.

What is claimed is:

1. An amphiphilic branched copolymer obtainable by an addition polymerisation process, wherein said polymer comprises:
at least two chains which are covalently linked by a bridge other than at their ends; and wherein the at least two chains comprise at least one ethylenically monounsaturated monomer, and wherein the bridge comprises at least one ethylenically polyunsaturated monomer; and wherein
the polymer comprises a residue of a chain transfer agent and optionally a residue of an initiator, and wherein
at least one of the monounsaturated monomer(s) and polyunsaturated monomer(s) and chain transfer agent(s) is a hydrophilic residue; and
at least one of one of the monounsaturated monomer(s) and polyunsaturated monomer(s) and chain transfer agent(s) is a hydrophobic residue, and wherein
the mole ratio of polyunsaturated monomer(s) to monounsaturated monomer(s) is in a range of from 1:100 to 1:4, and wherein
at least one of the monounsaturated monomer(s) and polyunsaturated monomer(s) and chain transfer agent(s) comprises a moiety that is capable of responding to an external stimulus thereby creating a physical or chemical change in the solubility of the said moiety; and wherein
the air-water surface tension of the polymer changes from between 35 mN/m to 60 mN/m upon application of the external stimulus.

2. The amphiphilic branched copolymer of claim 1 wherein the air-water surface tension of the polymer changes from between 40 mN/m to 55 mN/m upon application of an external stimulus.

3. The amphiphilic branched copolymer of claim 1 wherein the air-water surface tension of the polymer changes from between 42 mN/m to 52 mN/m upon application of the external stimulus.

4. The amphiphilic branched copolymer of claim 1 wherein the external stimulus is selected from: pH, ionic strength, sonic means, temperature, concentration, electromagnetic radiation or the addition of a further chemical entity.

5. The amphiphilic branched copolymer of claim 1 wherein one or more of the monounsaturated monomer(s), polyunsaturated monomer(s) and/or chain transfer agent(s) are each individually responsive to the external stimuli.

6. The amphiphilic branched copolymer of claim 1 wherein the residue of the chain transfer agent comprises between 0 to 80 mole % of the copolymer based on the number of moles of monofunctional monomer.

7. The amphiphilic branched copolymer of claim 1 wherein the residue of the chain transfer agent comprises between 0 to 50 mole % of the copolymer based on the number of moles of monofunctional monomer.

8. The amphiphilic branched copolymer of claim 1 wherein the residue of the chain transfer agent comprises between 0.05 to 30 mole % of the copolymer based on the number of moles of monofunctional monomer.

9. The amphiphilic branched copolymer of claim 1 comprising a monofunctional monomer which comprises one or more monomers selected from the group consisting of:
vinyl acids, vinyl acid esters, vinyl aryl compounds, vinyl acid anhydrides, vinyl amides, vinyl ethers, vinyl amines, vinyl aryl amines, vinyl nitriles, vinyl ketones, and derivatives thereof and corresponding allyl variants thereof; hydroxyl-containing monomers and monomers which may be post-reacted to form hydroxyl groups, acid-containing or acid-functional monomers; zwitterionic monomers and quaternised amino monomers; oligomeric, polymeric and di- and multi-functionalised monomers.

10. The amphiphilic branched copolymer of claim 9 wherein the monofunctional monomer are vinylic or allylic and are selected from the group consisting of styrenics, acrylics, methacrylics, allylics, acrylamides, methacrylamides, vinyl acetates, allyl acetates, N-vinyl amines, allyl amines, vinyl ethers, and allyl ethers.

11. The amphiphilic branched copolymer of claim 1 comprising a monofunctional monomer, wherein when the monofunctional monomer provides the necessary hydrophilicity in the copolymer, the monofunctional monomer is a residue of a hydrophilic monofunctional monomer, comprising a molecular weight of at least 1000 Daltons.

12. The amphiphilic branched copolymer of claim 1 comprising a multifunctional monomer which is selected from the group consisting of:
divinyl aryl monomers; (meth)acrylate diesters; polyalkylene oxide di(meth)acrylates;
divinyl (meth)acrylamides; divinyl ethers; and tetra- or tri-(meth)acrylate esters; vinyl or allyl esters, amides or ethers of pre-formed oligomers or polymers formed via ring-opening polymerisation, and oligomers or polymers formed via a living polymerisation technique such as oligo- or poly(1,4-butadiene).

13. A method of preparing the amphiphilic branched copolymer of claim 1 by an addition polymerisation process, which comprises the mixing together of:
i) at least one ethylenically monounsaturated monomer;
ii) from 1 to 25 mole % (based on the number of moles of monofunctional monomer(s)) of at least one ethylenically polyunsaturated monomer;
iii) a chain transfer agent; and
iv) an initiator, and subsequently reacting said mixture to form a branched copolymer.

14. The method of claim 13 wherein the addition polymerisation process comprises a free-radical polymerisation process.

15. The method of claim 13 wherein the initiator comprises a free-radical initiator.

16. An oil-in-water or water-in-oil emulsion comprising the amphiphilic branched polymer of claim 1 wherein the polymer is located at the oil-water interface.

17. The emulsion of claim 16, wherein the emulsion further comprises a dispersed phase and wherein an active ingredient is incorporated in the dispersed phase.

18. The emulsion of claim 16 wherein the average size of the droplets in the emulsion is less than 20 μm.

19. The use of the amphiphilic branched polymer of claim 1 as an emulsifier.

20. The use of the amphiphilic branched polymer of claim 1 as a triggered release agent.

21. An oil-in-water or water-in-oil emulsion comprising the amphiphilic branched polymer prepared by the method of claim 13, wherein the polymer is located at the oil-water interface.
